(19) United States
(12) Patent Application Publication
(10) Pub. No.: US 2005/0168149 A1
(43) Pub. Date: Aug. 4, 2005

(54) Flash lamp with high irradiance

(75) Inventors: Tatumi Hiramoto, Tokyo-to (JP); Yukihiro Morimoto, Himeji-shi (JP); Kazuyuki Mori, Himeji-shi (JP); Tetuya Torikai, Takasago-shi (JP)

Correspondence Address: NIXON PEABODY, LLP, 401 9TH STREET, NW, SUITE 900, WASHINGTON, DC, 20004-2128, US

(73) Assignee: Ushiodenki Kabushiki Kaisha, Tokyo (JP)

(21) Appl. No.: 11/042,165

(22) Filed: Jan. 26, 2005

(30) Foreign Application Priority Data
Jan. 29, 2004 (JP) 2004-021006

(51) Int. Cl. H01J 5/04

(52) U.S. Cl. 313/636

(57) ABSTRACT

A flash lamp with high radiation intensity which has a discharge vessel having a part in the region between the electrodes which has a smaller diameter than the inside diameter of the discharge vessel in the area in which the cathode is located, and the inner surface of the part of smaller diameter being provided with a heat-resistant material. A xenon gas is filled into the vessel at a room temperature pressure from 1.3×10³ Pa to 1.6×10⁵ Pa or a krypton gas is filled at 7×10² Pa to 1.3×10⁵ Pa. A power supply operates the flash lamp with a full width at half maximum of the current from 150 μs to 2 ms and a current density in the part with a small diameter of at least 2110 A/cm² when xenon is used and at least 2930 A/cm² when krypton is used.

### BACKGROUND OF THE INVENTION

1. Field of the Invention

The invention relates to a flash lamp filled with a rare gas such as xenon (Xe), krypton (Kr) and the like for emission. The invention relates especially to a flash lamp with high radiation density which is advantageously used for sterilization, curing of a photosensitive curing resin by UV radiation or visible radiation and for similar purposes.

2. Description of the Prior Art

Recently, a sterilization method using a flash lamp has been used because a flash lamp has the advantage that, by two mechanisms, specifically a photochemical mechanism which is used for sterilization by UV-C and UV-B radiation, and a photothermodynamic mechanism which is used to generate heat, a sterilization effect can be obtained. A flash lamp for sterilization purposes is described, for example, in Japanese patent disclosure document JP-A-2001-185088.

In order to increase the sterilization effect by the photochemical mechanism, it is, however, necessary for the radiation in the same region with wavelengths from 200 nm to 300 nm to have high radiation intensity. In a flash lamp which is used for sterilization, therefore, the flash light pulse width should be made smaller in order to increase the input power, and moreover, the current density larger than in a conventional flash lamp for optical heating which is used, for example, for fixing a toner in a printer and for similar purposes.

On the other hand, in a flash lamp, UV radiation in the region from 300 nm to 500 nm is effectively used for curing a photosensitive resin by UV radiation or visible radiation is possible. Specifically, the flash lamp is advantageously used for curing of an adhesive resin for purposes of cementing of disk elements in the production of digital versatile disks (DVD).

In the case of this curing of a photosensitive resin, radiation with a high radiation intensity in the wavelength region from 300 to 500 nm or shorter is obtained from a flash lamp operated with a high current density.

Such a flash lamp is used not only for this sterilization and this curing of a resin, but also for other purposes.

In a flash lamp which emits such radiation with a high radiance, however, the following disadvantages have increasingly appeared:
Since emission takes place in a state with a high current density, a high temperature state is caused. In this way, deterioration of the electrodes occurs prematurely.
The discharge vessel is subject to an enormous wall load. This causes premature deterioration of the discharge vessel.

This premature deterioration of the discharge vessel, of course, causes attenuation of the radiant light of the flash lamp and can lead to destruction of the discharge vessel in serious cases.

How a flash lamp with a high current density can be devised in which neither premature deterioration of the discharge vessel nor its destruction or the like occurs, and the optimum arrangement of this lamp, however, have never be adequately examined.

### SUMMARY OF THE INVENTION

A primary object of the present invention is to devise a flash lamp with high radiation intensity which has an arrangement in which a long service life can be achieved.

As a result of examining how a lamp with high radiation intensity can be attained, the following new findings not previously known were acquired:
The temperature can be increased by increasing the current density.
By reaching a high temperature, the thermal ionization of the rare gas for emission is accelerated, by which a large number of rare gas ions are formed.
By the mutual interaction of these ions with electrons, continuous radiation (emission spectrum) with high intensity is formed.
In addition, these rare gas ions are excited by thermal excitation.
As a result, ion emission radiation in the form of strong bright lines is obtained from the excited rare gas ions, by which the efficiency of conversion into radiation is increased.
Consequently a lamp with high radiation intensity can be obtained.

Lamp arrangements which are optimum for this were studied based on these findings.

An increase in the current density is theoretically possible by making the arrangement of the entire flash lamp smaller. However, it became apparent that, then, the size of the electrodes must also be reduced, that the temperature becomes high, that sputtering from the electrodes occurs and that the electrodes are worn off prematurely. This occurred more distinctly especially in the cathode with which the ions collide.

Therefore, to increase the current density the following was done, and as a result, it became possible to obtain radiation with high radiation intensity:
The inside diameter of the area of the discharge vessel in which the discharge takes place was made smaller and a narrow area was formed.
By constricting the discharge in a narrow area, the current density was increased, by which the temperature of the discharge area is increased, the discharge gas is ionized by thermal ionization and a high excitation state is produced by thermal excitation.

Since, in the electrodes, the electrode temperature for a small electrode becomes too high and since, in this way, electrode wear occurs to a major extent, a certain size of the electrodes and of the discharge vessel which surrounds the electrodes was ensured.

As a result it became apparent that it is advantageous if the discharge vessel has the following shape:

The middle has a small inside diameter.
The two ends, especially that of the cathode side, have a larger inside diameter than the middle.

The invention is described using the concept of “inside diameter”. The reason for using this expression is that the discharge vessel is generally produced using a round tube with a circular cross section. In the case of a discharge vessel with a special shape in which a round tube is not used, in which therefore the cross section is not round, it goes without saying that the size of the cross sectional area of the space in the discharge vessel is in question. Here, a smaller inside diameter means a small cross sectional area and a large inside diameter means a large cross sectional area.

By reducing the inside diameter of the discharge vessel in the area in which the discharge occurs, and by forming a part with a small diameter, the current density is increased. This leads to the possibility that the discharge vessel closely borders the discharge plasma, that the thermal load on the vessel increases and the disadvantages of milky opacification and destruction of the vessel and the like occur. Consideration of these disadvantages makes it necessary to arranged a heat-resistant component on the inside of part of the discharge vessel with a small diameter.

Specifically, the following was found.

By the arrangement of a pair of electrodes in the discharge vessel, by forming part of the discharge vessel between the electrodes with a smaller diameter than the inside diameter on the area of the discharge vessel in which the cathode is located, and by forming a surface which is in contact with the filling gas in the part with the small diameter from a heat-resistant material, a large amount of radiation is obtained in the wavelength range from 200 nm to 1000 nm.

A test was done to check in which region of the current density can radiation with high radiation intensity be obtained; in the test, a flash lamp filled with xenon gas was operated at the same operating wattage and by increasing the current density to 2110 A/cm² 2830 A/cm², 3390 A/cm² or the like. FIGS. 7, 8, & 9 show the spectral distribution of the radiation. In these drawings, the x-axis plots the wavelength of the radiation in the unit nm and the y-axis plots the relative radiation intensity without dimensions. In this way, the spectral distribution of the radiation is shown. These drawings indicate that around 200 nm to 300 nm and 400 nm to 600 nm radiation with high radiation intensity was obtained. It was found that in the invention the value of the current density must be greater than or equal to 2110 A/cm².

If the pressure of the xenon gas at room temperature is lower than 1.3×10³ Pa, the sputtering phenomenon begins to occur on the electrodes, by which premature wear of the electrodes occurs. This pressure of the xenon gas is therefore not realistic for a device. Furthermore, if the gas pressure at room temperature is higher than 1.6×10⁵ Pa, the trigger voltage during starting becomes high, by which operation becomes difficult and an operating circuit is required which has special electrical insulation in order to withstand this high voltage. This gas pressure is therefore unrealistic for a device. Therefore, it is necessary for the filling pressure of the xenon gas at room temperature to be in the range of 1.3×10³ Pa to 1.6×10⁵ Pa.

When the full width at half maximum of the current is less than 150 μs, the current width becomes too small, and a high wattage cannot be supplied. To supply high wattage, it is necessary to increase the peak current. However, when the peak current is increased, the sputtering phenomenon begins on the electrodes, by which the disadvantage of reducing the degree of radiation transmission of the discharge vessel occurs. When the full width at half maximum of the current is higher than 2 ms, it is necessary to ensure a capacitor capacitance which is enough to cause a corresponding large current to flow. This increases costs. This measure is therefore unrealistic. Therefore, operation with a full width at half maximum of the current from 150 μs to 2 ms must be carried out.

From the above described circumstance, the object is achieved in a flash lamp in accordance with the invention in that there is a pair of electrodes within a discharge vessel, that the discharge vessel is formed with a part between the electrodes that has a smaller diameter than the inside diameter of the discharge vessel in the area provided with the cathode, that the surface of the part with the small diameter which is in contact with the filling gas is made of a heat-resistant material, that only xenon gas or a mixed gas with xenon gas as the main component is added at room temperature with a pressure from 1.3×10³ Pa to 1.6×10⁵ Pa as the rare gas, and that the lamp is operated with a full width at half maximum of the current from 150 μs to 2 ms and a current density in the part with a small diameter of at least 2110 A/cm².

The case in which krypton gas is the filling gas is described below.

The formation of the part of the discharge vessel between the electrodes with a small diameter, the increase in the current density, and ensuring a certain size of the electrodes and of the discharge vessel surrounding the electrodes in the case in which the rare gas is krypton—exactly as in the case in which the gas is xenon—are likewise required. It is therefore advantageous for the discharge vessel to have the following shape:

The middle has a small inside diameter.
The two ends, especially that of the cathode side, have a larger inside diameter than the middle.

Since both xenon and krypton are rare gases and since the shapes of the distribution of the emission which is emitted in a discharge are similar, the value of the lower limit of the current density which is required in a flash lamp filled with krypton gas in order to obtain radiation with a high radiation intensity was determined based on the value of the current density of 2110 A/cm² in the case of a flash lamp filled with xenon. Specifically, when using the comparable expression that the energy supplied to the flash lamp and the energy emitted from the lamp are balanced, the Saha formula of thermal ionization for determining the electron density, the formula for determining the temperature based on the emission capacity of the krypton-filled flash lamp and the like, based on the value of the current density of the xenon-filled flash lamp in the state in which the ion effect begins, and based on the spectral distribution at this time, the value of the lower limit of the current density in a flash lamp filled with krypton is determined. This indicated that, to obtain radiation with high radiation density in a flash lamp filled with krypton gas, a current density of at least 2930 A/cm² in the part with the small diameter is necessary.

For the same reason as with xenon gas, the filling pressure of the krypton gas at room temperature must be 7×10² to 1.3×10⁵ Pa.

Furthermore, the full width at half maximum of the current must likewise be 150 μs to 2 ms.

From the above described circumstance, the object is achieved in a flash lamp in accordance with the invention in that there is a pair of electrodes within a discharge vessel, that the discharge vessel is formed between the electrodes with a part having a smaller diameter than the inside diameter of the discharge vessel in the area provided with the cathode, that the area of the part with the small diameter which is in contact with the filling gas is made of a heat-resistant material, that only krypton gas or a mixed gas with krypton gas as the main component is added as the rare gas with a pressure at room temperature of from 7×10² Pa to 1.3×10⁵ Pa, and that the lamp is operated with a full width at half maximum of the current from 150 μs to 2 ms and a current density in this part with a small diameter of at least 2930 A/cm².

The heat-resistant material with which the inside of the part with the small diameter is provided is advantageously a ceramic. When a radiation exit window is formed from this material with thermal resistance and translucency is required, it is more advantageous to use translucent aluminum oxide, non-transparent aluminum oxide, magnesium oxide (magnesia), yttrium oxide, YAG or aluminum nitride.

From the above described circumstance, the object is achieved in a flash lamp in accordance with the invention in that the above described heat-resistant material is a ceramic, among others translucent aluminum oxide, non-transparent aluminum oxide, magnesium oxide (magnesia), yttrium oxide, YAG or aluminum nitride.

Arranging a window or a radiation exit component of optical fiber on the tube axis of the discharge vessel makes it possible for pinched radiation to emerge with only little broadening.

The object is therefore achieved by the invention in a flash lamp in that at least one of the electrodes is located at a point which is remote from the tube axis of the discharge vessel, and that on the tube axis of the discharge vessel there is a radiation exit component.

Preferably, the electrode which is located at the point which is away from the tube axis is an anode, and the radiation exit component is located on the anode side of the discharge vessel on the tube axis. Thus, the cathode in which sputtering can often occur due to ion collision can be removed from the radiation exit window and has the effect that window fouling can be reduced.

Therefore, the object is achieved in accordance with the invention in a flash lamp in that the electrode which is located at the point which is away from the above described tube axis is an anode and that the radiation exit component is located on the anode side of the discharge vessel on the tube axis.

The object is furthermore achieved in accordance with the invention in a flash lamp in that the lamp can be used as the light source for a photochemical reaction and furthermore as the light source for a photochemical change and study of DNA and amino acids.

The object is therefore achieved according to the invention in a flash lamp in that the lamp can be used as a heat source for prompt surface heating using the property of high illuminance of the surface irradiation on the surface.

### ACTION OF THE INVENTION

By the flash lamp in accordance with the invention, a lamp with a high service life can be obtained in which the disadvantage of electrode wear does not occur, and furthermore, in which the radiation is converted into radiation with a high radiation intensity using ion emission and in which thermal deterioration of the discharge vessel hardly occurs.

The invention is further described below using several embodiments shown in the drawings.

### BRIEF DESCRIPTION OF THE DRAWINGS

FIG. 1 is a cross-sectional view of an embodiment of a flash lamp in accordance with the invention;

FIG. 2 is a cross-sectional view of a second embodiment of a flash lamp in accordance with the invention;

FIG. 3 is a cross-sectional view of a third embodiment of a flash lamp in accordance with the invention;

FIG. 4 is a cross-sectional view of a fourth embodiment of a flash lamp in accordance with the invention;

FIG. 5 is a cross-sectional view of a fifth embodiment of a flash lamp in accordance with the invention;

FIG. 6 shows an operating diagram of a flash lamp;

FIG. 7 shows a schematic of the spectral distribution of the radiation at a current density of 2110 A/cm³ of a flash lamp in accordance with the invention;

FIG. 8 shows a schematic of the spectral distribution of the radiation at a current density of 2830 A/cm³ of a flash lamp in accordance with the invention; and

FIG. 9 shows a schematic of the spectral distribution of the radiation at a current density of 3390 A/cm³ of a flash lamp in accordance with the invention;.

### DETAILED DESCRIPTION OF THE INVENTION

### Embodiment 1

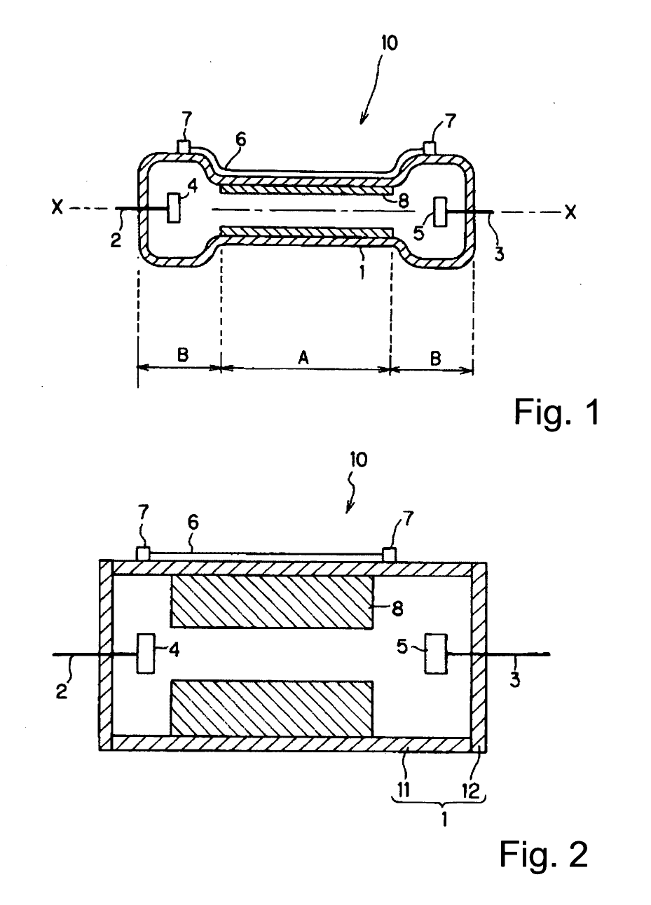

It is advantageous to reduce the inside diameter of the area of the discharge vessel in which the discharge takes place in order to increase the current density. FIG. 1 shows one example of a specific arrangement of this lamp.

A flash lamp 10 has a discharge vessel 1 which is present between the electrodes 4, 5 and which comprises a part A with a reduced inside diameter. The two ends B of the discharge vessel 1 are areas which are provided with electrodes and have a relatively great inside diameter. Consequently, the entire discharge vessel 1, in this example, is formed essentially in the form of a hand weight. Since, generally, a round tube is used for the discharge vessel 1, the middle of the part A with the small diameter, of the tube and of the two ends B on the tube axis X-X, and their cross sections are round. The cross-sectional shape, however, is not always limited to a circular shape. From the two ends of the discharge vessel 1, there extend electrode rods 2, 3 such that they project essentially on the tube axis in the direction to the inside of the discharge vessel 1. On the tips of the electrode rods 2, 3, on the one hand, the cathode 4 is formed, and on the other, the anode 5 is formed and they are disposed opposite each other in the discharge vessel 1. The coefficients of thermal expansion of the electrode rods 2, 3 and the discharge vessel 1 differ from one another. The vicinity of the electrode rods 2, 3 of the discharge vessel 1 is provided with a graded glass in which the glass is joined such that the coefficients of thermal expansion gradually change. The size of the electrodes 4, 5 was chosen such that, even when a high temperature arises due to high current density, no electrode wear by thermal load occurs. The inside diameter of the two ends B of the discharge vessel 1 is therefore greater than the part A with the small diameter of the middle.

The discharge of the flash lamp 10 arises between the electrodes 4, 5. Because the inside of the discharge vessel 1 in the area in which this discharge occurs is constricted and made narrow, the current density is increased. This increase of the current density by the constriction and narrowing of the inside leads to an increase of the thermal load of the discharge vessel 1, by which it is possible for the service life of the discharge vessel 1 to be shortened. As the material of the discharge vessel 1 generally, for example, silica glass or the like was used. However, in accordance with the invention, at least the inside of the part A of the discharge vessel with a small diameter is provided with a heat-resistant material. Ceramic is advantageously the heat-resistant material. Especially when radiant light emerges from the outside periphery of the part A with the small diameter, for the heat-resistant material translucency is required. It is therefore necessary to provide a layer 8 of translucent aluminum oxide, non-transparent aluminum oxide, magnesium oxide (magnesia), yttrium oxide, YAG or aluminum nitride. Specifically, there is a tube 8 of the above described material located on the inside of the silica glass discharge vessel 1 and which is made narrow. Furthermore, it goes without saying that the entire discharge vessel 1, including the tube 8, can also be produced from a heat-resistant material or it can be provided with it.

### Embodiment 2

In order to be able to place the part with a small diameter in the discharge vessel, the arrangement shown in FIG. 2 can also be undertaken. Here, another tubular component 8 is slipped onto the inside of the tube 11 of the discharge vessel and attached; its outside diameter is equal to the inside diameter of the tube. On the two ends of the tube 11, there is a cover 12 so that, overall, a cylindrical discharge vessel 1 is formed. In this case, the tube 8 must have a certain thickness on the inside in order to constrict the discharge part. The arrangement of the electrodes 4, 5 is identical to FIG. 1.

### Embodiment 3

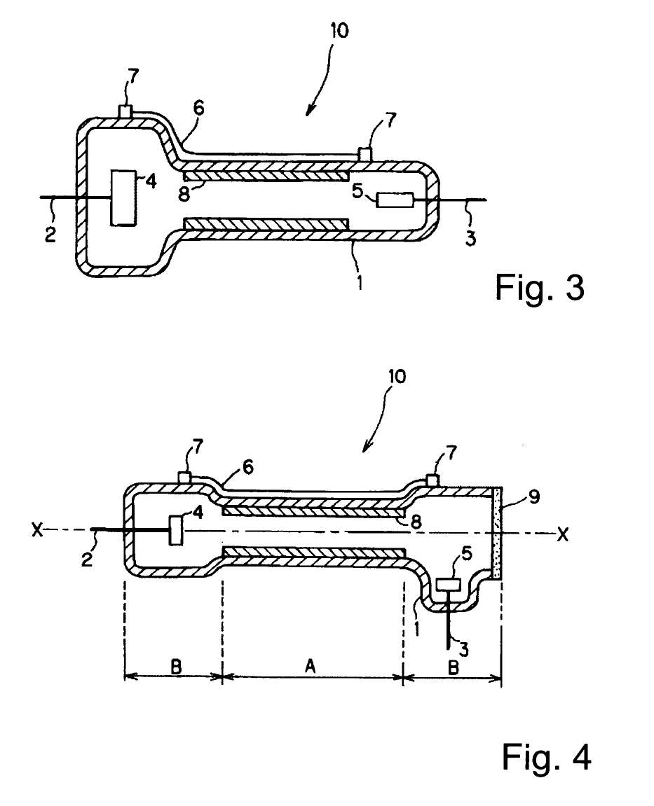

In the above described two examples, the electrode size of the anode 5 is essentially identical to the size of the cathode 4. However, this need not always be the case. It is disadvantageous for the cathode in which sputtering often takes place as a result of ion collision to be made small. However, the anode for which this disadvantage is minor can be made as a relatively small electrode. Therefore, as shown in FIG. 3, the diameter of the cathode 4 and the inside diameter of the discharge vessel 1 which surrounds the cathode 4, and thus, the cathode 4, can be made larger and also an arrangement of the discharge vessel 1 can be undertaken in which not only the inside diameter of the middle of the discharge vessel, but also the inside diameter of the discharge vessel area surrounding the anode 5, can be reduced.

In the embodiments 1 to 3 of FIGS. 1 to 3, there is a trigger electrode 6 running along the outside of the discharge vessel 1. A stop 7 for the trigger electrode 6 is located on the outside peripheral surfaces of the two ends of the discharge vessel 1.

The discharge vessel 1 is filled with xenon gas or krypton gas as the emission rare gas individually or as a gas mixture with xenon gas or krypton gas as the main component. In the case of a gas mixture with xenon gas as the main component, up to roughly 80 vol. % xenon gas can be added. The remainder of the gas mixture is krypton, argon, and/or neon. In the case of a gas mixture with krypton gas as the main component, roughly 80% krypton is added and the remainder of the gas mixture is xenon, argon and/or neon.

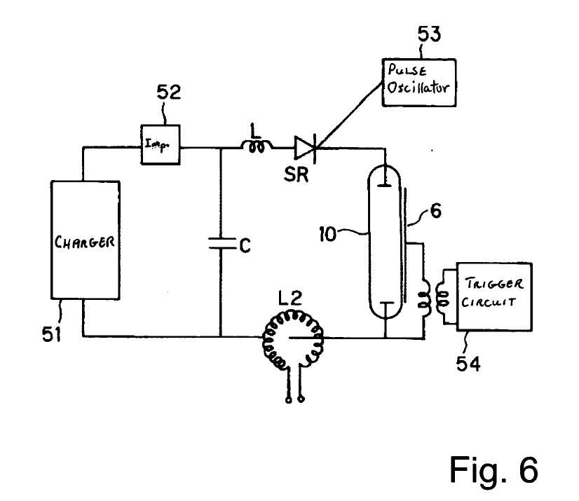

Because, for example, operation is carried out with the operation circuit described below and shown in FIG. 6, radiation in the wavelength range from 200 nm to 1000 nm takes place in the direction to the outside periphery of the part A with a small diameter of the discharge vessel 1. To irradiate the region with the large area or a large volume, it is advantageous to use this radiation which takes place from the outside surface of the part with a small diameter of the lamp with a high current density in the direction to the outside periphery.

FIG. 6 shows one example of an operation circuit of a flash lamp in accordance with the invention. A charging device 51 charges a capacitor C for charging and discharging via an impedance 52 for operation current control. A flash lamp 10 is series connected to a thyristor SR, an inductance element L and the capacitor C for charging and discharging. By transmitting ON-OFF signals from a pulse oscillator 53 to the thyristor SR, a discharge current from the capacitor C for charging and discharging is applied to the discharge lamp 10. On the other hand, essentially simultaneously with the ON signal from the pulse oscillator 53 of the trigger electrode 6, a trigger signal is sent from the trigger circuit 54, by which an insulation breakdown of the discharge space of the flash lamp 10 is induced. By this insulation breakdown, discharge current flows from the capacitor C for charging and discharging in the lamp, by which, in the flash lamp 10, a flash discharge takes place. A one-time flash emission takes place by this operation. This operation is repeated as necessary. When a Rogowski coil L2 is arranged in the circuit connected to the flash lamp 10 such that it surrounds the line between the capacitor C for charging and discharging and the flash lamp 10 and when the output voltage is measured, the current value can be determined (for example, see pp. 346 to 347 “14.1.2 magnetic probe” of Dec. 25, 1997 from the “University Lecture of the Electrogesellschaft, Plasma Technology”, collected and published by the Elektrogesellschaft). In this way, the voltage and the current are determined. When xenon is added as the gas, the voltage supplied to the flash lamp is changed such that the current density of the part with the small diameter of the flash lamp 10 is at least 2110 A/cm². By changing the capacitance of the inductance element L, the capacitance of the capacitor C and the like, the current density can also be changed. The current density is defined by the (maximum current value) divided by the (cross-sectional area of the part with the small diameter).

In the above described flash lamp 10, the middle area of the discharge vessel was made narrow, and thus, a narrowed part with a small diameter was produced. The inside diameter of the part with a small diameter and the current value of the flash lamp were chosen such that the current density in this part with a small diameter is at least 2110 A/cm². Furthermore, the arrangement of the flash lamp and the distance between the electrodes and the like, the capacitance L of the inductance element of the operation circuit, the capacitance of the capacitor C and the voltage supplied to the flash lamp and the like were chosen such that the full width at half maximum of the current was 150 μs to 2 ms.

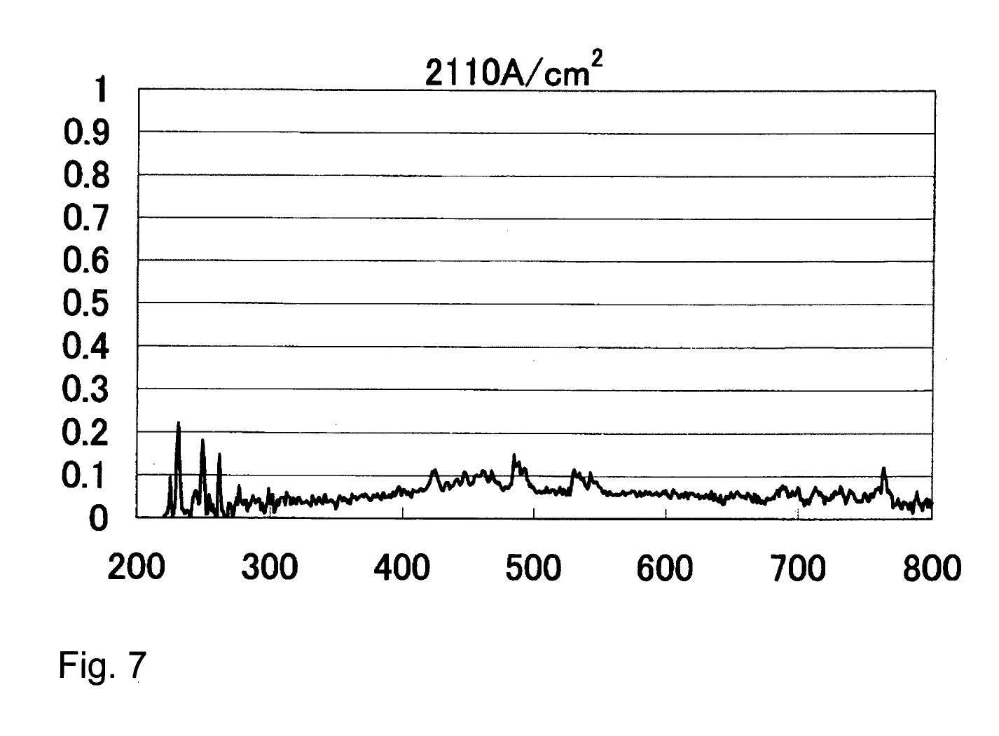

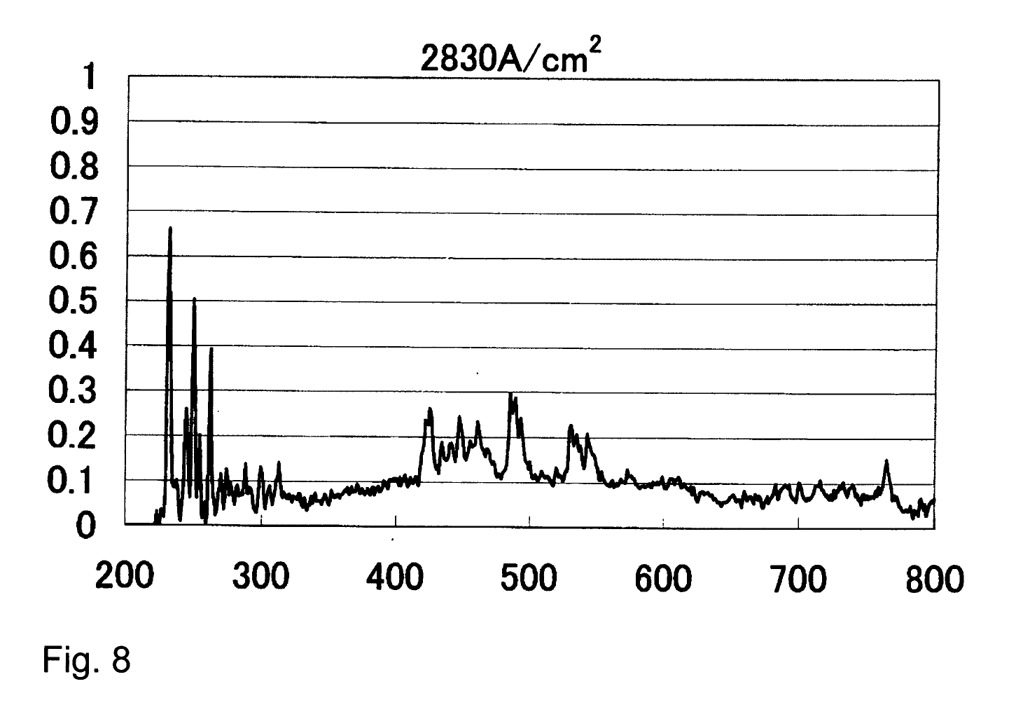

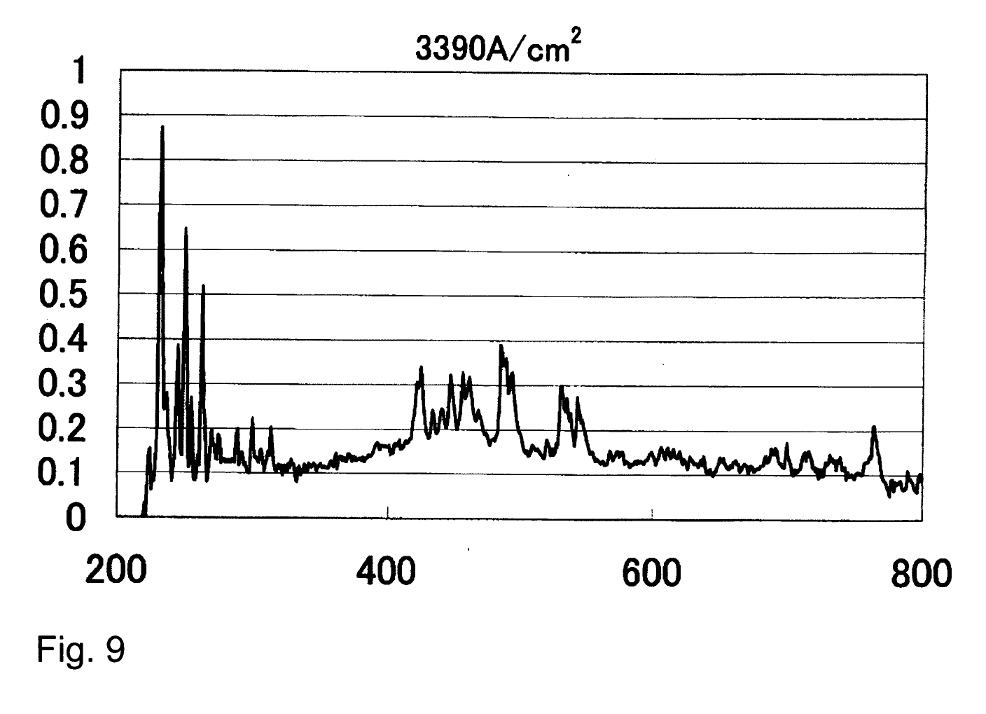

The inside diameter of the part with the small diameter of the discharge vessel was fixed at 3.5 mm and the inside diameter of the two ends of the discharge vessel was fixed at 8 mm. 1.3×10⁴ Pa xenon gas at room temperature were added to the discharge vessel, the current value was changed and the flash lamp was operated with current densities of 2110 A/cm², 2830 A/cm² and 3390 A/cm³. FIGS. 7, 8 & 9 show the spectral distribution of the radiation. As is apparent from the drawings, for the flash lamp of the invention, radiation in the range of from 200 nm to 1000 nm can be advantageously produced.

### Embodiment 4

In order to emit radiation with only little broadening from the discharge vessel at wavelengths from 200 nm to 1000 nm, a lamp with the arrangement shown in FIG. 4 can be used. The middle of the discharge vessel 1 has a part A with a reduced inside diameter. The two ends B of the discharge vessel 1 have a relatively large inside diameter and hold the electrodes. On the tube axis X-X of this discharge vessel 1, an electrode rod 2 extends such that it projects in the direction of the tube axis X-X to the inside. On the tip of the electrode rod 2 there is an electrode 4. The other electrode 5 is located on the tip of the electrode rod 3 which is arranged perpendicular to the tube axis X-X so that the electrode 5 is remote from the tube axis X-X of the discharge vessel.

On the tube axis X-X of the discharge vessel 1, there is a flat radiation exit window 9 so that radiation emerges. Since the discharge occurs on the tube axis X-X of the part A with a small diameter of the discharge vessel 1, by placing the radiation exit window 9 on the tube axis X-X, radiation with only little broadening can emerge. The material of the radiation exit window can be silica glass, sapphire, magnesium fluoride (MgF₂) or the like.

Since a vigorous sputtering phenomenon occurs in the cathode by collision of the ions which have been ionized by the discharge, and since the material sprayed off the cathode settles on the inside of the discharge vessel, and thus, milky opacification often occurs, the cathode as the electrode 4 is placed at a location remote from the radiation exit window 9 and the anode electrode 5 is removed from the tube axis X-X of the discharge vessel 1 and placed in the vicinity of the radiation exit window 9. This measure promises the action that the disadvantage of milky opacification of the inside of the discharge vessel 1 by sputtering from the cathode 4 can be avoided.

### Embodiment 5

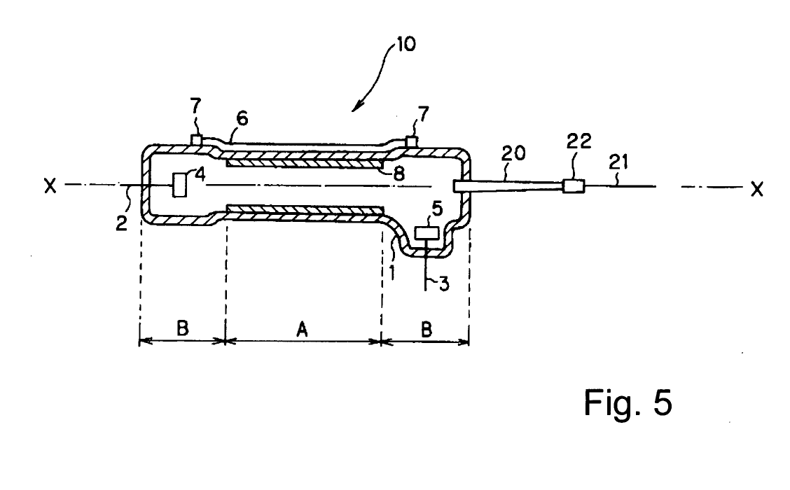

Furthermore, instead of the above described radiation exit window 9, there can be a light guide 20 with the function of radiation emergence on the tube axis X-X of the discharge vessel 1, and the radiation in this light guide 20 can be routed via a connector 22 to optical fibers 21 and thus emerge. FIG. 5 shows the arrangement of this flash lamp 10. Instead of the light guide 20 on the tube axis X-X of the discharge vessel 1, the optical fibers 21 can also be directly embedded.

To irradiate a region with a small area or a small volume it is advantageous to use the above described radiation which emerges from the light guide or from the radiation exit window in the direction of the tube axis.

The flash lamp in accordance with the invention can also be used for the following purposes.

The first application is for a light source for a photochemical reaction. For this, the following applications can be imagined, in addition to the above described curing of a resin.

(1) Ozone (O₃) effectively absorbs radiation with a wavelength range from roughly 220 nm to 290 nm and is decomposed, activated oxygen with a highly oxidizing action being produced. Therefore the following is done.

(1-1) By irradiation of an atmosphere which contains ozone with UV radiation, surface cleaning using ozone and UV radiation by using radiation in the above described wavelength range of the flash lamp of the invention is done. In doing so, the surface of the body which is to be cleaned is subjected to irradiation with the entire wavelength range. Radiation with a wavelength range which is absorbed by the body which is to be cleaned increases the surface temperature of the body which is to be cleaned. The decomposition of the ozone is also implemented by this high temperature. Thus, a greater cleaning action than only for simple decomposition of the ozone by UV radiation is developed.

(1-2) There is an increasing demand for an extreme reduction in the size of the gate oxide film of silicon (Si) or of a composed semiconductor. By irradiation of an atmosphere which contains ozone with UV radiation, activated oxygen forms as a result of decomposition of the ozone by the UV radiation. The article irradiated with lamp radiation, especially the area in which an oxide film is to be produced, can be heated. As a result, the activated oxygen diffuses more easily within the oxide film. This means that the rate of generation is high.

(2) By exposing the entire surface or only part of a resist for a KrF laser or an ArF laser which also has a photosensitive area in the wavelength range from 220 nm to 260 nm, before or after lithography exposure the flash lamp according to the invention can also be used in the curing of the resist and the like.

A second application relates to the photochemical change and determination of DNA and amino acids. For this purpose, besides the above described sterilization using the photochemical mechanism and the photothermodynamic mechanism, the following applications are possible.

(1) It is known that when DNA is irradiated with UV radiation with wavelengths from 200 nm to 280 nm mainly thymine which is one of the bases forms a dimer and is converted. Furthermore it was recognized that sterilization by death of bacteria takes place by this conversion of DNA. The UV resistance of the bacteria themselves depends on the resistance of the film surrounding the bacteria to UV radiation, especially on the translucency. Sterilization is carried out with UV radiation with wavelengths from 220 nm to 300 nm because in the area with wavelengths shorter than roughly 200 nm ozone is produced in the air. For sterilization by the radiation of the flash lamp the effect of the temperature increase of the bacteria by absorption of radiation in a range with longer wavelengths than the above described wavelengths occurs.

(2) In a living body only L-type amino acids occur. In artificial production, by using left-hand or right-hand circularly polarized radiation either only the L-type or only the D-type can be produced. By using circular polarization with a wavelength of roughly 220 nm, L-leucine (an essential amino acid) can be produced. In the wavelength range with 200 nm to 250 nm a changing composition of the essential amino acids is possible by a photochemical process by irradiation with circular polarization.

In this conversion of DNA and this production of amino acids radiation with certain wavelengths is absorbed. Using this circumstance, as a result of absorption of certain wavelengths, DNA and amino acids can be detected and analyzed. The flash lamp of the invention can also be used for this detection purpose.

In this way, the flash lamp in accordance with the invention can be used for a photochemical reaction, a photochemical change of DNA and proteins and for their determination.

Furthermore, the flash lamp according to the invention can be used for crystal healing after ion implantation into silicon (Si) or a composed semiconductor. The flash lamp of the invention can also be used for annealing a connecting area between bearings, a connecting area between a bearing and a substrate, and the like. Moreover, the flash lamp in accordance with the invention can also be used for surface heating for crystallization of amorphous silicon in a TFT liquid crystal display. In any case, heating only of the vicinity of the surface need be able to be done. In a flash lamp, the discharge time is short, and therefore, this heating can be achieved. In particular, since the reflection factor of the radiation with a wavelength of roughly at most 400 nm is small, heating with the flash lamp of the invention is advantageous, because it is rich in radiation in this wavelength range.

As was described above, the flash lamp in accordance with the invention can be used as a heat source for brief surface heating using the property of high illuminance of the irradiation on the surface.

1. Flash lamp with high irradiance, comprising:
a discharge vessel in which there is a pair of electrodes and which is filled with a gas which contains a rare gas, a region of the discharge vessel located between the electrodes having a part with a smaller diameter than an inside diameter of the discharge vessel in an area in which the cathode is located and
a power supply adapted for operating the flash lamp with a full width at half maximum of the current of from 150 μs to 2 ms and a current density in the part with a small diameter of at least 2110 A/cm²,
wherein a surface of the part with the smaller diameter which is in contact with said gas is formed essentially of a heat-resistant material, and
wherein the rare gas is added at room temperature with a pressure from 1.3×10³ Pa to 1.6×10⁵ Pa and comprises xenon or a gas mixture with xenon as the main component.

2. Flash lamp with high irradiance as claimed in claim 1, wherein the heat-resistant material is ceramic.

3. Flash lamp with high irradiance as claimed in claim 2, wherein the ceramic is selected from the group consisting of translucent aluminum oxide, non-transparent aluminum oxide, magnesium oxide (magnesia), yttrium oxide, YAG and aluminum nitride.

4. Flash lamp with high irradiance as claimed in claim 1, wherein at least one of the electrodes is located at a point which is remote from a longitudinal axis of the discharge vessel, and wherein a radiation exit component is provided on the longitudinal axis of the discharge vessel.

5. Flash lamp with high irradiance as claimed in claim 6, wherein the electrode which is located at the point which is away from the tube axis is an anode, and wherein the radiation exit component is located on the anode side of the discharge vessel.

6. Flash lamp with high irradiance, comprising:
a discharge vessel in which there is a pair of electrodes and which is filled with a gas which contains a rare gas, a region of the discharge vessel located between the electrodes having a part with a smaller diameter than an inside diameter of the discharge vessel in an area in which the cathode is located and
a power supply adapted for operating the flash lamp with a full width at half maximum of the current of from 150 μs to 2 ms and a current density in the part with a small diameter of at least 2930 A/cm²,
wherein a surface of the part with the smaller diameter which is in contact with said gas is formed essentially of a heat-resistant material, and
wherein the rare gas is added at room temperature with a pressure from 7×10² Pa to 1.3×10⁵ Pa and comprises krypton or a gas mixture with krypton as the main component.

7. Flash lamp with high irradiance as claimed in claim 6, wherein the heat-resistant material is ceramic.

8. Flash lamp with high irradiance as claimed in claim 7, wherein the ceramic is selected from the group consisting of translucent aluminum oxide, non-transparent aluminum oxide, magnesium oxide (magnesia), yttrium oxide, YAG and aluminum nitride.

9. Flash lamp with high irradiance as claimed in claim 6, wherein at least one of the electrodes is located at a point which is remote from a longitudinal axis of the discharge vessel, and wherein a radiation exit component is provided on the longitudinal axis of the discharge vessel.

10. Flash lamp with high irradiance as claimed in claim 6, wherein the electrode which is located at the point which is away from the tube axis is an anode, and wherein the radiation exit component is located on the anode side of the discharge vessel.

11. Method of a producing photochemical change and detecting or determining DNA and amino acids, comprising producing a photochemical reaction in a sample using a flash lamp with high irradiance, the flash lamp comprising:
a discharge vessel in which there is a pair of electrodes and which is filled with a gas which contains a rare gas, a region of the discharge vessel located between the electrodes having a part with a smaller diameter than an inside diameter of the discharge vessel in an area in which the cathode is located,
wherein a surface of the part with the smaller diameter which is in contact with said gas is formed essentially of a heat-resistant material, and
wherein the rare gas is added at room temperature with a pressure from 7×10² Pa to 1.3×10⁵ Pa and comprises krypton or a gas mixture with krypton as the main component or is added at room temperature with a pressure from 1.3×10³ Pa to 1.6×10⁵ Pa and comprises xenon or a gas mixture with xenon as the main component, and
further comprising the step of operating the flash lamp with power supplied with a full width at half maximum of the current of from 150 μs to 2 ms and a current density in the part with a small diameter of at least 2930 A/cm², if the rare gas is krypton and a current density in the part with a small diameter of at least 2110 A/cm² if the rare gas is xenon.

12. A method of heating a surface, comprising the steps of:
positioning a flash lamp with high irradiance in the vicinity of a surface to be heated, the flash lamp with high radiation density, the flash lamp comprising:
a discharge vessel in which there is a pair of electrodes and which is filled with a gas which contains a rare gas, a region of the discharge vessel located between the electrodes having a part with a smaller diameter than an inside diameter of the discharge vessel in an area in which the cathode is located,
wherein a surface of the part with the smaller diameter which is in contact with said gas is formed essentially of a heat-resistant material, and
wherein the rare gas is added at room temperature with a pressure from 7×10² Pa to 1.3×10⁵ Pa and comprises krypton or a gas mixture with krypton as the main component or is added at room temperature with a pressure from 1.3×10³ Pa to 1.6×10⁵ Pa and comprises xenon or a gas mixture with xenon as the main component, and
further comprising the step of operating the flash lamp with power supplied with a full width at half maximum of the current of from 150 μs to 2 ms and a current density in the part with a small diameter of at least 2930 A/cm², if the rare gas is krypton and a current density in the part with a small diameter of at least 2110 A/cm² if the rare gas is xenon.
